(19) United States
(12) Patent Application Publication
(10) Pub. No.: US 2018/0055988 A1
(43) Pub. Date: Mar. 1, 2018

(54) BLOOD CONDITION MONITOR, BLOOD CONDITION MONITORING METHOD, BLOOD CONDITION MONITORING SYSTEM, AND BLOOD CONDITION IMPROVING PROGRAM

(71) Applicant: SONY CORPORATION, TOKYO (JP)

(72) Inventor: MARCAURELE BRUN, TOKYO (JP)

(21) Appl. No.: 15/556,585

(22) PCT Filed: Feb. 12, 2016
(86) PCT No.: PCT/JP2016/054050

§ 371 Date: Sep. 7, 2017

(30) Foreign Application Priority Data
Mar. 20, 2015 (JP) 2015-057588

(51) Int. Cl. A61M 1/36; A61M 1/34

(52) U.S. Cl. CPC A61M 1/3639; A61M 1/3656; A61M 1/3403; A61M 1/3437; A61M 1/3441; A61M 1/3434

(57) ABSTRACT

Provided is a blood condition monitor using a permittivity-based coagulation measurement technology to monitor the condition of blood during extracorporeal circulation and thus being useful for avoiding blood problems from occurring during extracorporeal circulation. Provided is a blood condition monitor including: an extracorporeal circulation unit for extracorporeally circulating blood; and a first blood measurement unit for measuring an electrical characteristic of the blood obtained by applying an AC electric field to the blood. The first blood measurement unit is disposed in the blood circuit of the extracorporeal circulation unit. The blood condition monitor further includes a blood condition analysis unit for analyzing a change in blood condition on the basis of data on temporal changes in the electrical characteristic.

### TECHNICAL FIELD

The present invention relates to a blood condition monitor, a blood condition monitoring method, a blood condition monitoring system, and a blood condition improving program.

### BACKGROUND ART

Conventionally, extracorporeal circulation devices are used for artificial heart-lung machines, plasma exchangers, dialysis machines, and other applications.

Blood is known to have the property of forming rouleaux or thrombuses upon stimulation such as contact with materials other than vascular endothelial cells (foreign materials). Since extracorporeal circulation devices are made of artificial materials, allowing blood to flow through extracorporeal circulation devices increases the risk of, for example, thrombus formation. The occurrence of, for example, thrombus formation can not only hinder the extracorporeal circulation but also affect the patient.

In a conventional technology, the occurrence of, for example, thrombus formation during extracorporeal blood circulation is detected through fluctuations in pressure due to, for example, the clogging of a filter in an extracorporeal circulation device.

In addition, when an artificial heart-lung machine is used during cardiac surgery, for example, monitoring for thrombus formation is performed by, for example, periodically sampling blood every 30 to 60 minutes and measuring the activated clotting time. As a result of the monitoring, for example, in a case where thrombus formation is observed, the extracorporeal circulation device needs to be entirely replaced before the circulation is resumed.

Thus, how to prevent thrombus formation has been a problem with medical devices such as extracorporeal circulation devices.

For example, Patent Document 1 discloses development of a method of detecting thrombuses by the steps of sampling anticoagulant-treated fresh blood from a living body, removing erythrocytes from the fresh blood to form a blood product, cooling the blood product while circulating the blood product through a blood circulation circuit, neutralizing the anticoagulant, heating the blood product while applying laser sheet light to the blood product, and acquiring an image formed by scattered laser light.

In this method, thrombus precipitation is detected using a blood product obtained by removing erythrocytes from blood because in a case where laser light is applied to blood, it is not easy to distinguish between the signal from hemoglobin in the normal portion of blood and the signal from hemoglobin in the thrombus portion.

### CITATION LIST

### Patent Document

### Patent Document 1: Japanese Patent Application Laid-Open No. 2006-247200

### SUMMARY OF THE INVENTION

### Problems to be Solved by the Invention

Unfortunately, measurement methods used for blood coagulation monitoring in actual practice such as surgery are mechanical or optical measurement methods, which are all designed to measure the result of thrombus formation. At present, it is not possible to directly and continuously monitor the condition of whole blood during surgery or other procedures.

Additionally, for example, in the case of cardiac surgery, the possible need for replacement of the extracorporeal circulation device requires taking into account the time, expense, patient's burden, and other burdens required for the replacement.

It is a principal object of the present invention to provide a blood condition monitor using a permittivity-based coagulation measurement technology to monitor the condition of blood during extracorporeal circulation and thus being useful for avoiding blood problems from occurring during extracorporeal circulation.

### Solutions to Problems

As a result of extensive research to solve the above-mentioned object, the present inventor has succeeded in constantly monitoring a change in blood condition by using a permittivity-based coagulation measurement technology for the measurement of blood during extracorporeal circulation, and thus has completed the present technology.

Specifically, first, the present technology provides a blood condition monitor including:

an extracorporeal circulation unit for extracorporeally circulating blood; and

a first blood measurement unit for measuring an electrical characteristic of the blood obtained by applying an AC electric field to the blood.

In the blood condition monitor according the present technology, the first blood measurement unit may be disposed in the blood circuit of the extracorporeal circulation unit.

The blood condition monitor according to the present technology may further include a blood condition analysis unit for analyzing a change in blood condition on the basis of data on temporal changes in the electrical characteristic.

The blood condition monitor according to the present technology may further include a second blood measurement unit and a third blood measurement unit.

The blood condition monitor according to the present technology may further include a display unit for displaying at least one result selected from a result of measurement by the first blood measurement unit, a result of analysis based on data obtained from the first blood measurement unit, a result of measurement by the second blood measurement unit, a result of analysis based on data obtained from the second blood measurement unit, a result of measurement by the third blood measurement unit, and a result of analysis based on data obtained from the third blood measurement unit.

The blood condition monitor according to the present technology may further include a warning unit for issuing a warning in a case where a result of the analysis exceeds a predetermined blood condition criterion.

The blood condition monitor according to the present technology may further include a drug addition determination unit for determining whether or not to add a drug to the blood.

The blood condition monitor according to the present technology may further include a drug addition unit for adding a drug to the blood.

In addition, the blood condition may be a blood coagulation state, and the drug may be an anticoagulant.

The present technology is further directed to a blood condition device including:

a first blood measurement unit for measuring an electrical characteristic of blood obtained by applying an AC electric field to the blood; and a connection unit for connecting the blood measurement unit to an extracorporeal circulation unit for extracorporeally circulating the blood.

Next, the present technology provides a blood condition monitoring method including:

extracorporeally circulating blood;

measuring an electrical characteristic of the blood while applying an AC electric field to the blood; and

analyzing a change in blood condition on the basis of data on the measured electrical characteristic.

The present technology further provides a blood condition monitoring system including:

an extracorporeal circulation device for extracorporeally circulating blood; and

a measurement device for measuring an electrical characteristic of the blood obtained by applying an AC electric field to the blood,

the blood condition monitoring system being configured to analyze a change in blood condition on the basis of data on the measured electrical characteristic.

In the blood condition monitoring system of the present technology, the respective devices may be at least partially connected via a network.

In addition, the present technology also provides a blood condition improving program for causing a computer to execute a process including: analyzing a change in blood condition on the basis of data on an electrical characteristic of extracorporeally circulating blood obtained by applying an AC electric field to the blood to determine whether or not to add a drug to the blood; and adding the drug to the blood.

### Effects of the Invention

The present technology makes it possible to regularly or constantly monitor the condition of blood or an early sign of a change in blood while extracorporeally circulating the blood. In addition, the present technology also makes it possible to automatically take action, such as adding an anticoagulant or other drugs, which will eliminate, for example, the need for replacing the whole of an extracorporeal circulation device.

It will be understood that the effects described herein are non-limiting and the present technology may bring about any of the effects disclosed herein.

### BRIEF DESCRIPTION OF DRAWINGS

FIG. 1 is a schematic diagram showing an example of a first blood measurement unit of the present technology.

FIG. 2 is a schematic diagram showing an example of a first blood measurement unit of the present technology.

FIG. 3 is a schematic diagram showing an example of a second blood measurement unit of the present technology.

FIG. 4 is a schematic diagram showing an example of a blood condition monitor of the present technology.

FIG. 5 is a flow chart showing an example of a blood condition improving program of the present technology.

### MODE FOR CARRYING OUT THE INVENTION

Hereinafter, preferred modes for carrying out the present technology will be described. It will be understood that the embodiments described below are typical embodiments of the present technology and should not be construed as limiting the scope of the present technology. Note that descriptions will be provided in the following order.

1. Blood condition monitor
(1) Extracorporeal circulation unit
(2) First blood measurement unit
(3) Blood condition analysis unit
(4) Display unit
(5) Second blood measurement unit
(6) Third blood measurement unit
(7) Warning unit
(8) Drug addition determination unit
(9) Drug addition unit

2. Blood condition monitoring method

3. Blood condition monitoring system
(1) Extracorporeal circulation device
(2) Blood measurement device
(3) Blood condition analyzer and display of analysis

4. Blood condition improving program

5. First Embodiment

### 1. Blood Condition Monitor

(1) Extracorporeal Circulation Unit

The extracorporeal circulation unit as part of the present technology refers to a series of parts adapted to remove blood from a living body using a pump or other means, to circulate the blood through an extracorporeal blood circulation circuit, and to send the blood to the living body. Specifically, the extracorporeal circulation unit may be, for example, an artificial heart-lung machine, a hemodialysis machine, or a plasma exchanger.

In the present technology, an existing extracorporeal circulation circuit may be used without modification.

For example, the outline of the structure of the artificial heart-lung machine may be as follows.

First, the blood taken out of a living body is sent by a blood pump through a blood removal circuit to an artificial lung, where gas exchange is performed, and then sent to a blood feeding circuit.

A bubble removing device may be disposed upstream of the artificial lung, which the blood enters, and air may be trapped at the bubble removing device and then the blood may be sent to a blood reservoir.

Also, a reservoir may be disposed in the middle of the blood removal circuit. The reservoir is equipped with a vent and a myocardial protection vent pump for sucking excess blood from the heart and also equipped with a suction and a suction pump for collecting bleeding and returning it to the living body.

A myocardial protection circuit and a myocardial protection vent pump may also be provided to inject a myocardial protective liquid for stopping and protecting the heart.

Furthermore, a blood dilution unit may also be provided, which contains a diluent for adjusting the blood concentration. The dilution of the blood can be controlled according to the resulting hematocrit value measured by the blood measurement unit described below.

(2) First Blood Measurement Unit

In the present technology, the first blood measurement unit is a unit for measuring an electrical characteristic of blood obtained by applying an AC electric field to the blood. Preferably, the first blood measurement unit is a unit for measuring temporal changes in an electrical characteristic of blood.

The electrical characteristic of blood may be, for example, permittivity, impedance, admittance, capacitance, conductance, conductivity, or phase angle. These electrical characteristics can be converted to one another by the mathematical formulas shown in Table 1 below. Therefore, for example, the evaluation result obtained by evaluating the hematocrit value and/or the hemoglobin amount using the result of permittivity measurement of a blood sample will be the same as the evaluation result obtained using the result of impedance measurement of the same blood sample. Many of these electrical quantities and physical property values can be expressed using complex numbers, which will simplify the conversion formulas.

TABLE 1
<Major interchangeable electrical quantities and physical property values>

| Electrical quantities and | | |
|---|---|---|
| physical property values | Symbol | Complex number expression |
| Voltage | V | V* = \|V\| exp j (ωt + φ) |
| Current | I | I* = \|I\| exp j (ωt + φ) |
| Impedance | Z | Z* = R + jX (R: Resistance, X: |
| | | Reactance) |
| Admittance | Y | Y* = G + jB (G: Conductance, |
| | | B: Susceptance) |
| Capacitance | C | C* = C − jG/ω |
| Conductance | G | G* = G + jωC |
| Loss tangent | D or tanδ | |
| (Dielectric loss tangent) | | |
| Loss angle | δ | |
| Phase angle | θ | |
| Q value | Q | |
| Permittivity | ε | ε* = ε − jκ/ωε₀ |
| Conductivity | κ | κ* = κ + jωε₀ε |

<Mathematical formulas associating respective electrical
quantities and physical property values>

Z* = V*/I*
θ = φ − φ
Y* = 1/Z*
C = B/ω
D = tanδ = G/ωC = 1/Q
ε* = C*/C₀
κ* = jωε₀ε*
ω: Angular frequency
ε₀: Vacuum permittivity (constant)
C₀: Constant depending on measurement device or other factors
Values with *: Complex numbers

The first blood measurement unit may be disposed at any position of the extracorporeal circulation unit, and is not particularly limited in the present technology. The blood circuit of the extracorporeal circulation unit may be branched, and the first blood measurement unit may be disposed at the branch. Preferably, however, the first blood measurement unit should be disposed in the blood circuit of the extracorporeal circulation unit. For example, if the extracorporeal circulation unit is the artificial heart-lung machine, the first blood measurement unit may be disposed in the blood removal circuit or the blood feeding circuit or in both the blood removal circuit and the blood feeding circuit.

In the present technology, therefore, blood measurement is successfully performed while the blood is extracorporeally circulated without being sampled, in contrast to the convention technique in which blood is sampled in tubes or other containers and then subjected to blood tests.

In a case where the temperature of the blood being fed is adjusted in the artificial heart-lung machine or the like, the first blood measurement unit is preferably disposed at a position where the blood can be measured immediately after the temperature adjustment. This is because, immediately after the temperature adjustment, variations in the temperature of the blood to be measured are small, so that variations in the measurement will also be small.

In addition, the first measurement unit is preferably disposed at a position where the blood can be measured after the removal of bubbles. This is because in such a case, the blood measurement can be performed without being affected by bubbles.

Further, one or more first blood measurement units may be provided.

The first blood measurement unit is, for example, generally configured to have a sample introduction section for introducing, as an analyte, the blood circulating in the extracorporeal circulation unit, in which the sample introduction section is placed in the blood circuit of the extracorporeal circulation unit. The sample introduction section may be, for example, but not limited to, what is called a sample cartridge. A pair of electrodes is inserted in the sample introduction section, in which the blood flows between the pair of electrodes. An AC electric field is applied to the blood in the sample introduction section by applying an AC voltage from a power source to the electrodes.

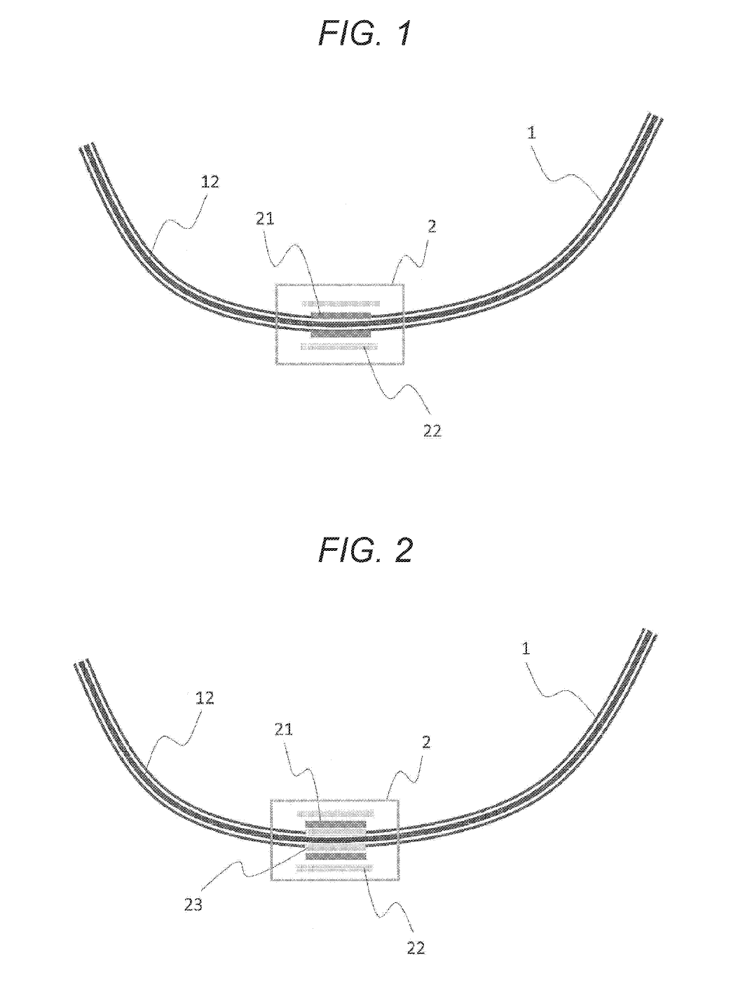

FIG. 1 shows an example of the first blood measurement unit. The blood circuit 1 of the extracorporeal circulation unit, in which the blood 12 flows from left to right in FIG. 1, is provided with the first blood measurement unit 2. The blood measurement unit 2 has, in its inside, a pair of electrodes 21 and an electrode cover 22 covering the electrodes 21. The blood 12 flows between the pair of electrodes and comes in direct contact with the electrodes 21.

The electrical characteristic of the blood can be measured even when the electrodes are in direct contact with the blood. However, the electrodes may be covered with, for example, a biocompatible plastic film with a thickness that does not impair the effects of the present technology. This makes it possible to suppress the formation of, for example, thrombuses. FIG. 2 shows an example of the first blood measurement unit in which the electrodes are covered with a biocompatible plastic film. In contrast to the case shown in FIG. 1, a biocompatible plastic film 23 is placed inside the electrodes 21 to prevent the electrodes 21 from being in direct contact with the blood 12.

Regarding the voltage, an AC voltage at a predetermined frequency may be applied at preset measurement intervals to the electrodes, or may be continuously applied to the electrodes so that the measurement can be constantly performed. In this way, an AC electric field at a predetermined frequency is applied to the blood.

The frequency band for use in the electrical measurement may be appropriately selected according to the condition of the blood to be measured, the purpose of the measurement, or other factors. For example, in a case where the electrical characteristic to be measured is impedance, changes can be observed in the frequency bands shown in Table 2 below according to changes in blood condition.

TABLE 2

| | Impedance | |
|---|---|---|
| | | Frequency at which |
| Change in blood | Frequency at which | change is more |
| condition | change is observable | significant |
| Blood coagulation | 1 kHz to 50 MHz | 3 MHz to 15 MHz |
| (blood clotting) | | |
| Fibrin formation | 1 kHz to 50 MHz | 3 MHz to 15 MHz |
| Fibrin clot formation | 1 kHz to 50 MHz | 3 MHz to 15 MHz |
| Blood clot formation | 1 kHz to 50 MHz | 3 MHz to 15 MHz |
| Erythrocyte rouleaux | 500 kHz to 25 MHz | 2 MHz to 10 MHz |
| formation | | |
| Blood agglutination | 1 kHz to 50 MHz | 500 kHz to 5 MHz |
| Erythrocyte | 1 kHz to 50 MHz | 100 kHz to 40 MHz |
| sedimentation (blood | | |
| sedimentation) | | |
| Clot retraction | 1 kHz to 50 MHz | 10 kHz to 100 kHz |
| (retraction) | | |
| Hemolysis | 1 kHz to 50 MHz | 3 MHz to 15 MHz |
| Fibrinolysis | 1 kHz to 50 MHz | 3 MHz to 15 MHz |

For example, for blood coagulation, the impedance is preferably measured at a frequency of 1 kHz to 50 MHz, more preferably at a frequency of 3 MHz to 15 MHz. Thus, a parameter may be selected in advance according to, for example, the blood condition so that the preferred frequency band can be automatically selected as shown in Table 2 above.

It will be understood that in the present technology, any test item for the blood may be appropriately selected and measured using the first blood measurement unit, and the first blood measurement unit is not specifically limited in the present technology. Examples of test items include those related to the blood coagulation system, such as the hematocrit value and blood clotting ability. More specifically, the measurement to be performed may be instantaneous measurement of the blood during extracorporeal circulation, measurement to determine a more coagulable state, or measurement to determine whether coagulation has begun.

For example, in the first blood measurement unit, a dielectric spectrum at 500 kHz to 10 MHz may be measured every 5 seconds. Since the blood is circulated though the first blood measurement unit, the blood differs when measured every 5 seconds. Also, since the blood is circulated, erythrocyte rouleaux formation would be less likely to occur. Thus, for example, an increase in the permittivity at 1 MHz or 10 MHz can be regarded as reflecting blood agglutination, suggesting blood coagulation.

As the hematocrit value increases, the permittivity at 1 MHz or 10 MHz increases like the case of blood agglutination. For example, the permittivity at 2 MHz may be used to distinguish between an increase in permittivity due to an increase in hematocrit value and an increase in permittivity due to blood agglutination. The permittivity at 2 MHz hardly changes due to blood coagulation or blood agglutination, but changes with the hematocrit value. Specifically, a case where the permittivity does not change at 2 MHz but increases at 1 MHz or 10 MHz can be regarded as an early stage of blood coagulation. In addition, a case where the permittivity increases at 2 MHz, 1 MHz, and 10 MHz can be regarded as indicating a change in the hematocrit value.

(3) Blood Condition Analysis Unit

The blood condition analysis unit is configured to analyze a change in blood condition on the basis of data on the electrical characteristic of the blood. Preferably, the blood condition analysis unit is configured to make an analysis on the basis of data on temporal changes in the electrical characteristic of the blood.

For example, in a case where the electrical characteristic of the blood is impedance, a change in blood condition can be analyzed by the following procedure.

First, on the basis of the impedance measured over time, the permittivity is calculated from known functions or relational expressions as described above.

The permittivity is known to increase as erythrocytes agglutinate. Therefore, the start of agglutination of erythrocytes can be known by determining whether or not the permittivity exceeds a predetermined threshold value (reference value).

In addition, the permittivity data obtained at predetermined intervals may be divided by the reference permittivity before the agglutination of erythrocytes to obtain ratios, so that temporal changes in the permittivity can be observed, reflecting the early stage of blood coagulation reaction.

Furthermore, a database or parameters indicating the correlation between the rate of permittivity change and the risk of thrombus formation may be obtained in advance and used to determine that the risk of thrombus formation is high when the rate of permittivity change exceeds a predetermined value. It is possible to know the trend of the blood coagulation system at an early stage and to know whether or not thrombus or rouleaux formation can easily occur even before thrombus or rouleaux formation occurs. In addition, other data such as the hematocrit value may also be used in combination with the permittivity to make the evaluation.

In this regard, the measurement and analysis of the blood condition may be performed with reference to, for example, the blood coagulation system analysis apparatus, blood coagulation system analysis method, and program described in Japanese Patent Application Laid-Open No. 2010-181400, the blood coagulation system analysis method and blood coagulation system analysis apparatus described in Japanese Patent Application Laid-Open No. 2012-194087, and the blood coagulation system analysis apparatus, the blood coagulation system analysis method, and the program therefore described in Japanese Patent Application Laid-Open No. 2013-221782.

(4) Display Unit

The result of the analysis may be displayed on a display unit such as a display or a print.

Besides the result of the analysis, examples of information that may be displayed on the display unit include conditions such as the flow rate, rate of dilution, and temperature of the blood in the extracorporeal circulation unit, the normal/abnormal state of the device in the blood condition measurement unit, the result of measurement by the second blood measurement unit described below, the result of analysis of the data obtained from the second blood measurement unit, the result of measurement by the third blood measurement unit described below, the result of analysis of the data obtained from the third blood measurement unit, the warning about the blood condition, the result of determination on whether to perform drug addition, and the amount of addition of the drug.

(5) Second Blood Measurement Unit

The second blood measurement unit is a unit for performing a test on an item appropriately selected from various blood test items other than the item measured with the first blood measurement unit.

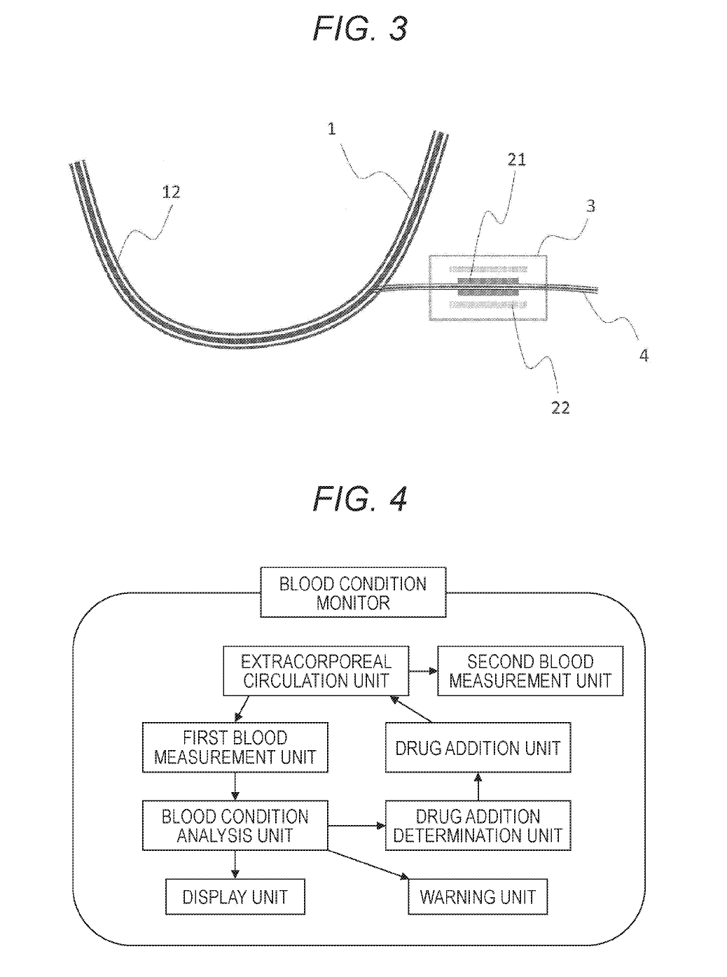

The second blood measurement unit may be disposed in the blood circuit of the extracorporeal circulation unit, or the blood may be branched from any desired part of the blood circuit and subjected to the second blood measurement outside the blood circuit of the extracorporeal circulation unit. For example, the configuration shown in FIG. 3 may be used. The blood circuit 1 of the extracorporeal circulation unit, in which the blood 12 flows from left to right in FIG. 3, is provided with a blood circuit 4, which is branched from the blood circuit 1 and directed to the downstream side. The branched blood circuit 4 is provided with a second blood measurement unit 3. If the blood is not returned to the blood circuit 1 after measured by the second blood measurement unit 3, the electrodes may be in direct contact with the blood and thus may easily cause, for example, thrombus formation. Therefore, electrodes 21 and an electrode cover 22 are provided in the example of FIG. 3, where the electrodes 21 do not have to be covered with a biocompatible plastic film or other materials.

Alternatively, instead of being branched, the blood may be directly sampled from the blood circuit 1 and then subjected to the measurement. After subjected to the measurement, the blood may be discarded as it is.

In addition, one or more second blood measurement units may be provided.

In the second blood measurement unit, the test may be performed on any item. For example, a coagulant such as Ca or TF may be added to the blood, and then the blood coagulation process may be measured. The coagulation time may be calculated from the coagulation process and then used to evaluate or test the risk of thrombus formation. Alternatively, the risk of thrombus formation may be evaluated or tested by adding, to the blood, aspirin, a prostaglandin preparation, a thromboxane synthase inhibitor, a platelet inhibitor such as cytochalasin D, a fibrinolytic system promoter such as a plasminogen activator, a fibrinogen function inhibitor such as H-Gly-Pro-Arg-Pro-OHxAcOH (Pefabloc FG), a fibrin polymerization inhibitor, a fibrinolytic system inhibitor such as a plasmin inhibitor such as aprotinin or tranexamic acid, a coagulation inhibitor such as heparin, or an inhibitor such as heparin, and extracting a parameter related to the strength of blood coagulation.

The data from the second blood measurement unit may be used for checking or assisting the data obtained from the first blood measurement unit and/or the result of analysis by the blood condition analysis unit.

The data from the second blood measurement unit may be analyzed by the blood condition analysis unit, or an additional analysis unit may be provided for the second blood measurement unit.

In addition, a program may also be created in advance for determining the blood condition by associating the data obtained from the second blood measurement unit and/or the result of the analysis thereof with the result of the first blood measurement analysis obtained from the blood condition analysis unit.

For example, when it is determined from the data from the first blood measurement unit that the blood coagulation is at an early stage, the blood may be sampled in the second blood measurement unit, mixed with Ca as a coagulant, and subjected to blood coagulation measurement. In a case where the resulting blood coagulation time is shorter than the reference value, it can be determined that the coagulation has definitely progressed, and the determination information may be sent to, for example, the warning unit described later.

Alternatively, besides Ca, any other coagulant may be added to the sampled blood, which may then be subjected to the measurement under similar conditions, so that a comparison can be made between the results obtained using Ca and obtained using the other anticoagulant, which will make it possible to evaluate the efficacy of the anticoagulant.

Alternatively, aspirin, a prostaglandin preparation, a thromboxane synthase inhibitor, a platelet inhibitor such as cytochalasin D, a fibrinolytic system promoter such as a plasminogen activator, a fibrinogen function inhibitor such as H-Gly-Pro-Arg-Pro-OHxAcOH (Pefabloc FG), a fibrin polymerization inhibitor, a fibrinolytic system inhibitor such as a plasmin inhibitor such as aprotinin or tranexamic acid, a coagulation inhibitor such as heparin, or an inhibitor such as heparin may be added to the sampled blood, which may then be subjected to blood coagulation measurement.

(6) Third Blood Measurement Unit

The third blood measurement unit is a unit for performing a test on an item appropriately selected from various blood test items other than the items measured by the first and second blood measurement units.

The third blood measurement unit may be disposed in the blood circuit of the extracorporeal circulation unit, or the blood may be branched from a desired part of the blood circuit and subjected to the third blood measurement outside the blood circuit of the extracorporeal circulation unit. Alternatively, the blood may be directly sampled from the blood circuit. After subjected to the measurement, the blood may be discarded as it is.

In addition, one or more third blood measurement units may be provided.

In the third blood measurement unit, the test may be performed on any item. Examples of the test include tests on items related to the blood coagulation system, tests on platelet count, erythrocyte count, hemoglobin, hematocrit value, prothrombin time, activated partial thromboplastin time, and fibrinogen, hepaplastin test, and ATIII test.

A detailed blood test can be performed when an item other than the test items for the first and second blood measurement units is measured by the third blood measurement unit.

For example, the concentration of a specific blood coagulation factor may be measured, which makes it possible to identify the cause of initiation of blood coagulation (such as the influence of extracorporeal blood circulation or the influence of surgery).

The data from the third blood measurement unit may be used for checking or assisting the data and analysis result obtained from the first and/or second blood measurement unit.

The data from the third blood measurement unit may be analyzed by the blood condition analysis unit, or an additional analysis unit may be provided for the third blood measurement unit.

In addition, a program may also be created in advance for determining the blood condition by associating the data obtained from the third blood measurement unit and/or the result of the analysis thereof with the result of the first and/or second blood measurement analysis obtained from the blood condition analysis unit.

(7) Warning Unit

The blood condition monitor of the present technology may include a warning unit. The warning unit is a unit for issuing a warning in a case where, for example, the data from the first, second, or third blood measurement unit or the result of analysis of the data exceeds a predetermined blood condition criterion.

The warning may be displayed on the display unit or may be issued in any other form such as a sound.

Whether or not to issue the warning is determined on the basis of the analysis by the blood condition analysis unit.

For example, in a case where the electrical characteristic of the blood is impedance, the warning unit may be configured to issue the warning, for example, when the permittivity of the blood increases, when the permittivity exceeds a predetermined threshold, when certain temporal changes in the permittivity are observed reflecting the early stage of blood coagulation reaction, or when the rate of change in the permittivity exceeds a predetermined value, in which a database or parameters indicating the correlation between the rate of permittivity change and the risk of thrombus formation are obtained in advance.

When the warning is issued, an operation to prevent/inhibit blood coagulation should preferably be performed immediately.

(8) Drug Addition Determination Unit

The drug addition determination unit is a unit for determining whether or not to add a drug to the blood on the basis of, for example, the data from the first, second, or third blood measurement unit, the result of analysis by the blood condition analysis unit, or the warning issued by the warning part.

For example, in a case where the electrical characteristic of the blood is impedance, the drug addition determination unit determines whether or not to add a drug to the blood, for example, when the permittivity of the blood increases, when the permittivity exceeds a predetermined threshold, when certain temporal changes in the permittivity are observed reflecting the early stage of blood coagulation reaction, or when the rate of change in the permittivity exceeds a predetermined value, in which a database or parameters indicating the correlation between the rate of permittivity change and the risk of thrombus formation are obtained in advance.

(9) Drug Addition Unit

The blood condition monitor of the present technology may include a drug addition unit. The drug addition unit is a unit for adding a drug to the blood, for example, on the basis of the data from the first, second, or third blood measurement unit, the result of analysis by the blood condition analysis unit, or the warning issued by the warning portion, or when the drug addition determination unit determines that the drug should be added to the blood.

The drug may be added in a manner depending on, for example, the type of the drug, the administration method suitable for the drug, the drug amount based on the weight/blood volume of the living body, the concentration and dose of the drug, or the administration rate.

For example, an anticoagulant is added when it is determined from the data from the blood condition analysis unit that there is an early sign of thrombus formation. This makes it possible to prevent thrombus formation by adding the drug before thrombus formation in contrast to a conventional method of adding the drug after thrombus formation.

The anticoagulant may be, for example, undifferentiated heparin, low molecular weight heparin, nafamostat mesilate, or argatroban.

The drug addition unit may include, for example, a drug storing section, a drug concentration adjusting section, and a section for introducing the drug into the blood circuit of the extracorporeal circulation.

The drug addition unit may be disposed at any desired location of the extracorporeal circulation unit. For example, the drug addition unit may be disposed at a blood feeding circuit immediately upstream of the place where the blood returns to the living body.

In addition, one or more drug addition units may be provided. A plurality of drug addition units may be provided to finely control the drug addition.

The addition of the drug may be followed by measuring the blood by the first, second, or third blood measurement unit, evaluating the effect of the addition of the drug, and adjusting the amount of addition of the drug on the basis of the evaluation result.

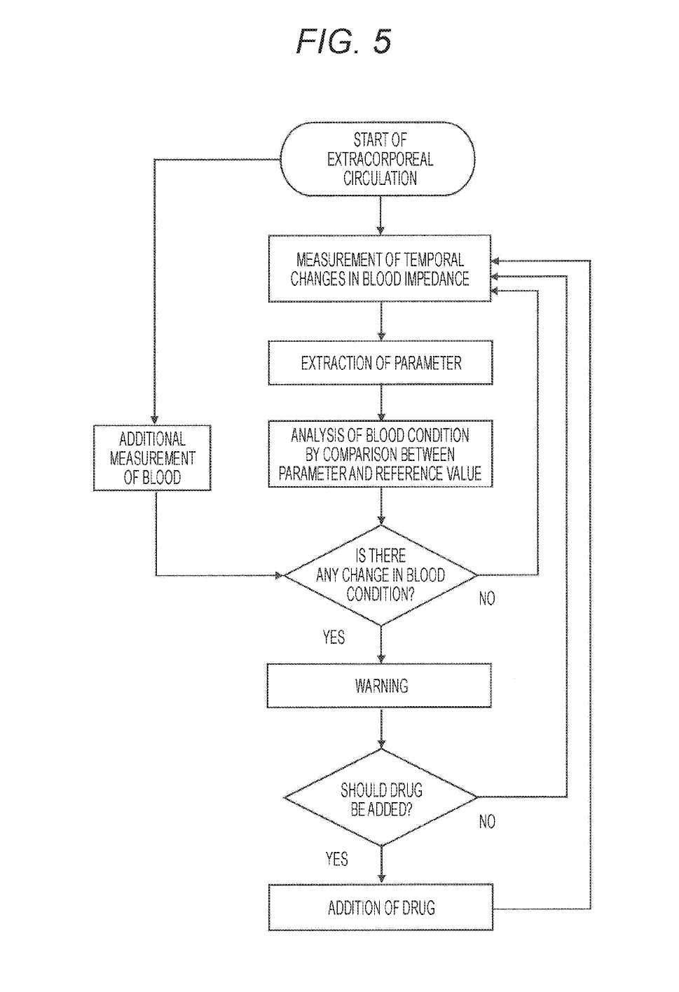

In this regard, FIG. 4 shows an example of the relationship among the above respective units in the blood condition monitor of the present technology.

Additionally, in an embodiment, the blood condition monitor of the present technology may include the first blood measurement unit for measuring an electrical characteristic of blood obtained by applying an AC electric field to the blood; and a connection unit for connecting the blood measurement unit to an extracorporeal circulation unit for extracorporeally circulating the blood.

According to the present technology, for example, the first blood measurement unit may be connected to a conventional artificial heart-lung machine with the connection unit placed between them so that the blood can flow from the heart-lung machine to the first blood measurement unit. For example, the connection unit may have a structure in which the first blood measurement unit is connected to the tube of the blood circuit in such a way that the circulating blood will not leak out.

### 2. Blood Condition Monitoring Method

In the present technology, the blood condition may be monitored by a process that includes extracorporeally circulating the blood, measuring an electrical characteristic of the blood while applying an AC electric field to the blood, and analyzing a change in blood condition on the basis of data on the measured electrical characteristic of the blood.

The electrical characteristic of the blood is measured by the first blood measurement unit.

For example, while an AC voltage at a specific frequency is applied to the blood circulating in the extracorporeal circulation unit, an electrical characteristic of the blood is measured over time, so that data on temporal changes in the electrical characteristic of the blood are obtained.

For example, if the electrical characteristic of the blood is impedance, the permittivity can be calculated as described above from the impedance data using known functions or relational expressions.

Next, for example, the state of coagulation is evaluated from the temporal permittivity data by estimating the coagulability of the blood on the basis of the range of fluctuations in the blood coagulation time. If the fluctuation range is relatively small, it can be determined, for example, that the blood is in a coagulable state or has started to coagulate.

The criterion for the evaluation to be performed may be, for example, data obtained immediately after the extracorporeal blood circulation is started, blood data already known to indicate the normal blood condition, or data on a standard model of blood.

Subsequently, when the rate of change in the permittivity exceeds a predetermined value, it can be determined, for example, that the risk of thrombus formation is high.

In addition, in the monitoring of the blood condition, the data obtained from the second or third blood measurement unit may be used as confirmatory or auxiliary reference data.

### 3. Blood Condition Monitoring System

The blood condition monitoring system of the present technology includes an extracorporeal circulation device for extracorporeally circulating blood and a blood measurement device for measuring an electrical characteristic of blood obtained by applying an AC electric field to the blood, and is configured to analyze a change in the blood condition on the basis of data on the measured electrical characteristic of the blood. The analysis can be performed by a blood condition analyzer.

(1) Extracorporeal Circulation Device

As mentioned above, the extracorporeal circulation device may be, for example, an artificial heart-lung machine, a hemodialysis machine, or a plasma exchanger. In the present technology, an existing extracorporeal circulation circuit may be used without modification.

(2) Blood Measurement Device

For example, if the electrical characteristic of the blood is impedance, the blood measurement device may be, for example, but not limited to, an existing impedance measurement device (such as an impedance analyzer (4294 A) manufactured by Agilent Technologies).

Here, a configuration example in a case where the blood condition measurement device is a device for measuring the blood coagulation system will be described below.

The device for measuring the blood coagulation system includes a sample cartridge into which blood is allowed to flow, a pair of electrodes for applying an AC voltage to the blood allowed to flow into the sample cartridge, a power source for applying an AC voltage to the electrodes, and a measurement unit for measuring the permittivity of the blood.

The measurement unit may include, for example, a signal processing section for outputting the measurement result to the blood condition analysis unit.

The sample cartridge may be provided with a drug introduction port for adding, for example, an anticoagulant to the blood.

The power source is configured to apply a voltage at the time of receiving an instruction to start the measurement or at the time when the power is turned on, in which the time is designated as a starting point. Specifically, the power source is configured to apply an AC voltage at a predetermined frequency to the electrodes constantly or at preset measurement intervals.

The measurement unit is configured to measure an electrical characteristic of the blood between the electrodes at a predetermined cycle at the time of receiving an instruction to start the measurement or at the time when the power is turned on, in which the time is designated as a starting point, and also configured to derive the permittivity from the measured value. As mentioned above, the permittivity is derived using known functions or relational expressions indicating the relationship between electrical characteristics and the permittivity.

(3) Blood Condition Analyzer and Display of Analysis

Data indicating the permittivity derived from the measurement unit is given at each measurement time to the blood condition analyzer. Upon receiving the permittivity data given from the measurement unit, the blood condition analyzer starts, for example, to determine the coagulability of the blood. The blood condition analyzer allows the result of determination of the coagulability and/or the permittivity data to be displayed or printed, for example, in the form of a graph, on a display or a predetermined medium.

In this regard, the blood measurement device, the blood condition analyzer, the display device, and other devices may be partially or entirely connected via a network.

### 4. Blood Condition Improving Program

The blood condition improving program of the present technology causes a computer to execute a process including: measuring an electrical characteristic of extracorporeally circulating blood obtained by applying an AC electric field to the blood; analyzing the blood condition on the basis of data on the measured electrical characteristic to determine whether or not to add a drug to the blood; and adding the drug to the blood.

The blood condition improving program of the present technology can be implemented, for example, according to the flow chart shown in FIG. 5.

First, the extracorporeal circulation is started, and an electrical characteristic of blood, such as temporal changes in impedance, is measured in the extracorporeal circulation circuit.

On the basis of the impedance measured over time, the permittivity is calculated from known functions or relational expressions, and a parameter representing the characteristic of the permittivity is extracted from the data on the temporal changes in the permittivity.

The blood condition is analyzed by comparing the extracted parameter with a predetermined reference value.

As a result, in a case where a change in the blood condition is observed (YES), a warning is issued, and it is determined whether or not to add a drug. In a case where no change in the blood condition is observed (NO), the blood measurement is continued.

Next, in a case where it is determined to add a drug (YES), the drug is added to the blood. In a case where it is determined that no drug should be added (NO), the blood measurement is continued.

Note that, in the series of flows, any desired blood test other than the measurement of the electrical characteristic of blood may be performed as second or third blood measurement. The result of this blood test may be incorporated as a reference into the determination of a change in the blood condition in the series of flows.

The blood condition improving program of the present technology is recorded on an appropriate recording medium.

### 5. First Embodiment

Hereinafter, a representative embodiment of the present technology will be described.

The blood from a living body is circulated through a blood circuit. The first blood measurement unit is disposed most upstream of the blood circuit. The blood is allowed to flow into the sample cartridge of the first blood measurement unit, in which an AC voltage is applied to the blood when temporal changes in electrical characteristic are measured. The measurement result is sent to the blood condition analysis unit, in which the permittivity is calculated. From the permittivity, analysis is performed to determine whether there is an early sign of blood coagulation, and the result is displayed on the display unit. In the warning unit, it is determined whether or not to issue the warning according to the result, and when issued, the warning is sent to the drug addition determination unit. The drug addition determination unit determines whether or not to add an anticoagulant to the blood.

On the other hand, the second and third blood measurement units are disposed downstream of the first blood measurement unit. In the second blood measurement unit, a coagulant is added to the blood sampled from the blood circuit, and the blood coagulation time is measured. In the third blood measurement unit, the fibrinogen concentration of the blood sampled from the blood circuit is measured. These measurement results are displayed on the display unit.

In accordance with the result of determination by the drug addition determination unit, the drug addition unit adds an anticoagulant to the circulating blood.

Note that the present technology may also have the following configurations.

[1] A blood condition monitor including:

an extracorporeal circulation unit for extracorporeally circulating blood; and

a first blood measurement unit for measuring an electrical characteristic of the blood obtained by applying an AC electric field to the blood.

[2] The blood condition monitor according to item [1], in which the first blood measurement unit is disposed in a blood circuit of the extracorporeal circulation unit.
[3] The blood condition monitor according to item [1] or [2], further including a blood condition analysis unit for analyzing a change in blood condition on the basis of data on temporal changes in the electrical characteristic.
[4] The blood condition monitor according to any one of items [1] to [3], further including a second blood measurement unit.
[5] The blood condition monitor according to item [4], further including a third blood measurement unit.
[6] The blood condition monitor according to item [5], further including a display unit for displaying at least one result selected from a result of measurement by the first blood measurement unit, a result of analysis based on data obtained from the first blood measurement unit, a result of measurement by the second blood measurement unit, a result of analysis based on data obtained from the second blood measurement unit, a result of measurement by the third blood measurement unit, and a result of analysis based on data obtained from the third blood measurement unit.
[7] The blood condition monitor according to any one of items [3] to [6], further including a warning unit for issuing a warning in a case where a result of the analysis exceeds a predetermined blood condition criterion.
[8] The blood condition monitor according to any one of items [1] to [7], further including a drug addition determination unit for determining whether or not to add a drug to the blood.
[9] The blood condition monitor according to any one of items [1] to [8], further including a drug addition unit for adding a drug to the blood.
[10] The blood condition monitor according to any one of items [1] to [9], in which the blood condition is a blood coagulation state.
[11] The blood condition monitor according to any one of items [8] to [10], in which the drug is an anticoagulant.
[12] A blood condition monitor including:

a first blood measurement unit for measuring an electrical characteristic of blood obtained by applying an AC electric field to the blood; and

a connection unit for connecting the blood measurement unit to an extracorporeal circulation unit for extracorporeally circulating the blood.

[13] A blood condition monitoring method including:

extracorporeally circulating blood;

measuring an electrical characteristic of the blood while applying an AC electric field to the blood; and

analyzing a change in blood condition on the basis of data on the measured electrical characteristic.

[14] A blood condition monitoring system including:

an extracorporeal circulation device for extracorporeally circulating blood; and

a measurement device for measuring an electrical characteristic of the blood obtained by applying an AC electric field to the blood,

the blood condition monitoring system being configured to analyze a change in blood condition on the basis of data on the measured electrical characteristic.

[15] The blood condition monitoring system according to claim 14, in which the respective devices are at least partially connected via a network.
[16] A blood condition improving program for causing a computer to execute a process including: analyzing a change in blood condition on the basis of data on an electrical characteristic of extracorporeally circulating blood obtained by applying an AC electric field to the blood to determine whether or not to add a drug to the blood; and adding the drug to the blood.

### REFERENCE SIGNS LIST

1 Blood circuit
2 First blood measurement unit
3 Second blood measurement unit
4 Branched blood circuit
12 Blood
21 Electrode
22 Electrode cover
23 Biocompatible plastic film

1. A blood condition monitor comprising:
an extracorporeal circulation unit for extracorporeally circulating blood; and
a first blood measurement unit for measuring an electrical characteristic of the blood obtained by applying an AC electric field to the blood.

2. The blood condition monitor according to claim 1, wherein the first blood measurement unit is disposed in a blood circuit of the extracorporeal circulation unit.

3. The blood condition monitor according to claim 1, further comprising a blood condition analysis unit for analyzing a change in blood condition on the basis of data on temporal changes in the electrical characteristic.

4. The blood condition monitor according to claim 1, further comprising a second blood measurement unit.

5. The blood condition monitor according to claim 4, further comprising a third blood measurement unit.

6. The blood condition monitor according to claim 5, further comprising a display unit for displaying at least one result selected from a result of measurement by the first blood measurement unit, a result of analysis based on data obtained from the first blood measurement unit, a result of measurement by the second blood measurement unit, a result of analysis based on data obtained from the second blood measurement unit, a result of measurement by the third blood measurement unit, and a result of analysis based on data obtained from the third blood measurement unit.

7. The blood condition monitor according to claim 3, further comprising a warning unit for issuing a warning in a case where a result of the analysis exceeds a predetermined blood condition criterion.

8. The blood condition monitor according to claim 1, further comprising a drug addition determination unit for determining whether or not to add a drug to the blood.

9. The blood condition monitor according to claim 1, further comprising a drug addition unit for adding a drug to the blood.

10. The blood condition monitor according to claim 1, wherein the blood condition is a blood coagulation state.

11. The blood condition monitor according to claim 8, wherein the drug is an anticoagulant.

12. A blood condition monitor comprising:
a first blood measurement unit for measuring an electrical characteristic of blood obtained by applying an AC electric field to the blood; and
a connection unit for connecting the blood measurement unit to an extracorporeal circulation unit for extracorporeally circulating the blood.

13. A blood condition monitoring method comprising:
extracorporeally circulating blood;
measuring an electrical characteristic of the blood while applying an AC electric field to the blood; and
analyzing a change in blood condition on the basis of data on the measured electrical characteristic.

14. A blood condition monitoring system comprising:
an extracorporeal circulation device for extracorporeally circulating blood; and
a measurement device for measuring an electrical characteristic of the blood obtained by applying an AC electric field to the blood,
the blood condition monitoring system being configured to analyze a change in blood condition on the basis of data on the measured electrical characteristic.

15. The blood condition monitoring system according to claim 14, wherein the respective devices are at least partially connected via a network.

16. A blood condition improving program for causing a computer to execute a process comprising: analyzing a change in blood condition on the basis of data on an electrical characteristic of extracorporeally circulating blood obtained by applying an AC electric field to the blood to determine whether or not to add a drug to the blood; and adding the drug to the blood.
